(19) United States
(12) Patent Application Publication
(10) Pub. No.: US 2025/0090779 A1
(43) Pub. Date: Mar. 20, 2025

(54) ASSEMBLY FOR CONTROLLING THE NASAL DELIVERY OF A PRODUCT, AND ASSOCIATED DELIVERY SYSTEM AND CONTROL METHOD

(71) Applicant: Nemera La Verpillière, LA VERPILLIERE (FR)

(72) Inventors: Alain REGARD, BEYNOST (FR); Xavier JOURDAN, LE PASSAGE (FR); Lenaic GUIMARD, CHARAVINES (FR); Thierry DECOCK, LYON (FR)

(73) Assignee: Nemera La Verpillière, LA VERPILLIERE (FR)

(21) Appl. No.: 18/970,373

(22) Filed: Dec. 5, 2024

Related U.S. Application Data
(63) Continuation of application No. PCT/EP2023/065442, filed on Jun. 9, 2023.

(30) Foreign Application Priority Data
Jun. 10, 2022 (FR) FR2205636

(51) Int. Cl. A61M 15/08

(52) U.S. Cl. CPC A61M 15/085; A61M 2205/215; A61M 2205/332

(57) ABSTRACT

The invention relates to an assembly (1) for controlling the dispensing of a product into a nasal cavity of a user, comprising:
a first holder (20) configured to be fastened to a product dispensing device (30) for nasal pathway, and comprising a first orientation sensor (21) configured to measure a data relating to the orientation of the first holder (20) in space,
a second holder (10) configured to be fastened to the user's head, and comprising a second orientation sensor (11) configured to measure a data relating to the orientation of the second holder (10) in space along three reference axes, and
an information processing system suitable for receiving the data measured by the first sensor (21) and the second sensor (11) and for determining an information about the orientation of the first holder (21) with respect to the second holder according to said measured data.

The invention relates to the technical field of nasal dispensing of a product, more precisely into the nasal cavity, more particularly near the olfactory region located on the upper side of the nasal cavity, near the brain.

The state of the art, in particular in document WO 2021/014076 A1, already describes an assembly for controlling the dispensing of a product comprising a remote mobile device fastened to the user's ear and an orientation sensor associated with a nasal dispensing device, one of the objectives being to determine the position of the nasal dispensing device with respect to the remote mobile device. According to this document, the mobile device also comprises an orientation sensor to determine its position in space. However, the mobile device proposed in this document is fastened to the ear, whose auricle may have different dimensions from one user to another. Thus, fastening the mobile device to the user's ear can pose stability and repeatability difficulties. The position of this mobile device can in fact vary from one use to another, therefore the position of this mobile device with respect to the user's nose can also vary, so there is a non-negligible risk that the nasal dispensing device is not positioned with respect to the user's nose at the optimum angles which would allow better product dispensing in a target area, for example that is remote and difficult to access such as the olfactory region of the nasal cavity.

The invention aims in particular to optimise, during its use, the determination of the orientation of the device for the nasal dispensing of a product with respect to the user's nose in particular to optimise the positioning of the dispensing device to ensure efficient product dispensing.

This good positioning is particularly important when dispensing medications aimed at treating in particular diseases such as Alzheimer's, Parkinson's, or even autism, mood or satiety disorders.

Thus, the invention relates to an assembly for controlling the dispensing of a product into a nasal cavity of a user comprising:
a first holder configured to be fastened to a product dispending device for nasal pathway and comprising a first orientation sensor configured to measure a data relating to the orientation of the first holder in space,
a second holder configured to be fastened to the user's head and comprising a second orientation sensor configured to measure a data relating to the orientation of the second holder in space along three reference axes, and
an information processing system configured to receive the data measured by the first orientation sensor and the second orientation sensor and to determine an information about the orientation of the first holder with respect to the second holder according to said measured data, the second holder being configured to be fastened to the user's forehead or nose, so that a first plane formed by two of the reference axes of the second orientation sensor is located, when the second holder is fastened to the user's head, in a predetermined orientation with respect to a sagittal plane of the user, preferably with respect to the sagittal plane of the user's head, or with respect to a frontal plane of the user, preferably with respect to the frontal plane of the user's head.

The second holder is placed on the user's forehead or nose, so that the positioning of the second orientation sensor is reliable, stable and repeatable during the various uses. Thus, such an arrangement minimises the dependency of the positioning or of the orientation of the second sensor on the user's anatomy or on how it is placed in/on a user's ear.

The characteristic “the second holder being configured to be fastened to the user's forehead or nose” also includes the case where the second holder is fastened to both the user's forehead and nose.

Predetermined orientation means a known orientation that is substantially the same each time the second holder is fastened to the user's forehead or nose. It is therefore the structure of the second holder and how it is fastened to the user's forehead or nose, following a user guide for example, which makes it possible to obtain the predetermined orientation. It is therefore possible to deduce from the orientation of the first holder with respect to the second holder, the orientation of the first holder with respect to the frontal or sagittal plane of the user since the orientation of the second holder with respect to the frontal or sagittal plane is known. In addition, since the position of the nose with respect to the sagittal and frontal planes of the user is known, it is therefore possible to determine the orientation of the first holder and therefore of the dispensing device in the user's nostril. The position of the nose is fixed with respect to the frontal and sagittal planes of the user's head.

“A first holder configured to be fastened to a product dispensing device” means a first holder adapted to be fastened in motion to the product dispensing device. A movement in space of the product dispensing device results in a similar movement of the first holder and therefore of the first sensor. Thus, the orientation measured by the first sensor fastened to the first holder is representative of the orientation of the dispensing device. However, in some embodiments, the first holder is removably fastened to the product dispensing device.

“A second holder configured to be fastened to the user's head” means a second holder adapted to be fastened in motion to the user's head. A movement in space of the user's head results in a similar movement of the second holder and therefore of the second sensor.

The second holder indicates the orientation of the head, therefore of the user's nose. We therefore obtain a reliable and repeatable reference frame to detect the orientation of the first holder with respect to the second holder, therefore with respect to the sagittal and/or frontal planes of the user, and preferably to the sagittal and/or frontal planes of the user's head, since the second holder is mounted in a precise orientation on the user's head. Since the first holder is configured to be fastened to the dispensing device, the orientation of the first holder represents that of the dispensing device. Thus, the orientation of the dispensing device in the user's nostril can be determined more reliably and more precisely.

It is particularly advantageous that the second holder is fastened to the user's forehead or nose rather than in or on an ear. The ear is in fact a flexible organ, and consequently the position and orientation of a sensor in/on an ear is less reliable and repeatable than on the nose or forehead.

The determination of the information about the orientation of the first holder with respect to the second holder and in particular the measurement of the so-called relative angles between the first orientation sensor and the second orientation sensor is relevant to evaluate whether the dispensing device is correctly positioned in one of the nostrils before activating the dispensing of the product. More particularly, the fact that a plane formed by two reference axes of the second orientation sensor is, due to the structure of the second holder and its positioning on the user's forehead or nose by the user themselves or by a practitioner, arranged in a predetermined manner, preferably parallel, with respect to the sagittal or frontal plane of the user, preferably with respect to the sagittal or frontal plane of the user's head, allows easier and more precise measurement of the orientation of the dispensing device and advantageously an orientation of the dispensing device during its use that is particularly stable and repeatable and, in particular, that depends very little on the anatomy of the user.

Precise determination of the positioning of the dispensing device with respect to the nose offers several advantages. This precise determination helps, for example, to identify during “user studies” during the development phase of a dispensing device, the dispensing device which makes it possible to obtain the positioning required for more efficient product dispensing in the target area when activating the device. In other use contexts, such as for example a clinical study or home use, this also helps to guide the user to position the dispensing device at the angles required for more efficient product dispensing in the target area. This good positioning is particularly important when dispensing medications aimed at treating diseases such as Alzheimer's, Parkinson's, or even autism, mood or satiety disorders.

The sagittal plane of a user corresponds to the plane that divides the body into a right part and a left part. The sagittal plane of the user's head corresponds to the plane that divides the head into a right part and a left part. The frontal plane of the user, also sometimes called the coronal plane, corresponds to a plane perpendicular to the sagittal plane of the user and separates the body into an anterior or ventral part and a posterior or dorsal part. The frontal plane of the user's head corresponds to a plane perpendicular to the sagittal plane of the user's head and separates the head into an anterior part and a posterior part. The nasal septum, separating the two nostrils of a user, is located substantially in the sagittal plane. The first holder comprises means for fastening to the dispensing device which can be permanent or removable fastening means. The advantage of removable fastening means is that the assembly for controlling the dispensing of a product can be used to control the orientation of different types of dispensing device which include, for example, different dispensing heads. The assembly for controlling the dispensing of a product is therefore preferably reusable whereas the dispensing device and in particular its dispensing head can be single-use.

The information processing system can communicate with the first sensor and the second sensor by wire. The first sensor and the second sensor can also each cooperate with the processing system via a wireless communication module. The processing system can be, for example, a smartphone or a microcontroller, for example embedded on the second holder or on the first holder.

The first orientation sensor and the second orientation sensor are each, for example, an inertial measurement unit comprising at least one accelerometer and one gyroscope. The three axes of the accelerometer, also called the reference axes of the corresponding sensor, advantageously form an orthonormal reference frame. The gyroscope can be used to measure the angular speed on three axes which are advantageously the three axes of the accelerometer of the same inertial measurement unit. Optionally, the inertial measurement unit further comprises a magnetometer used to improve the measurement precision of the inertial measurement unit.

According to other optional but not mandatory characteristics of the invention, the assembly for controlling the dispensing of a product comprises one or more of the following characteristics, taken alone or in any technically possible combination:
the predetermined orientation of the first plane corresponds to an orientation parallel to the sagittal plane of the user or to the frontal plane of the user.

Thus, thanks to the second holder configured this way, the second sensor is mounted fixed and parallel to the sagittal plane or the frontal plane on the user's head, so that the orientation of the dispensing device is known in reference to the sagittal or frontal plane of the user. We therefore obtain a reliable and repeatable reference frame to detect the orientation of the first holder with respect to the second holder and therefore with respect to the user's nostril. In addition, it is therefore easier to determine the orientation of the first holder with respect to the sagittal or frontal plane of the user and therefore with respect to the user's nostril.

The predetermined orientation of the first plane corresponds to an orientation parallel to the sagittal plane of the user's head or to the frontal plane of the user's head. We therefore obtain a fixed reference frame with respect to the user's nostril regardless of the orientation of the user's head.

The second holder is configured so that, once fastened to the user's forehead or nose, the second sensor is located above the user's nose, between the eyes or at the centre of the forehead.

The second holder is configured to be fastened to the user's forehead or nose, so that the first plane is located in a predetermined orientation with respect to the sagittal plane of the user and a second plane, formed by two of the reference axes of the second orientation sensor and different from the first plane, is located in a predetermined orientation with respect to the frontal plane of the user.

More generally, the user or a practitioner can position the second holder on the user's forehead or nose and the structure of the second holder then makes it possible to position the second holder so that the first plane and the second plane are respectively in their predetermined orientations with respect to the sagittal and frontal planes of the user, in other words preferably respectively parallel to the sagittal plane and parallel to the frontal plane. It is therefore possible to obtain a reference frame that is even more reliable and more repeatable to detect the orientation of the first holder with respect to the second holder, therefore with respect to the user's nostril since the orientations of the second holder with respect to both the sagittal plane and the frontal plane are known.

The second holder is configured to be fastened to the user's forehead or nose, so that the first plane is located in a predetermined orientation with respect to the sagittal plane of the user's head and a second plane, formed by two of the reference axes of the second orientation sensor and different from the first plane, is located in a predetermined orientation with respect to the frontal plane of the user's head.

It is therefore possible to obtain a reference frame independent of the orientation of the user's head.

The second holder is configured to be fastened to both the user's forehead and nose. This further increases the immobilisation of the second holder with respect to the user's head and therefore the reliability and repeatability of the measurement of the orientation of the product dispensing device.

The second orientation sensor comprises an accelerometer configured to measure the linear acceleration along three measurement axes, the measurement axes corresponding to the reference axes of the second sensor.

It is therefore easier to determine the orientation of the first sensor with respect to the sagittal and/or frontal planes of the user, preferably with respect to the sagittal and/or frontal planes of the user's head.

The second holder comprises a fastening device on the user's head, a base for receiving the second orientation sensor, adjustment elements for adjusting the position of the base with respect to the fastening device and locking elements for locking the position of the base so that the position of the second orientation sensor with respect to the sagittal plane of the user, preferably with respect to the sagittal plane of the user's head, can be adjusted and locked once the fastening device is fastened to the head of a user, the adjustment elements preferably comprising pivoting elements for pivoting the base about a pivoting axis perpendicular to the sagittal plane of the user, preferably to the sagittal plane of the user's head.

The fastening device can be used in particular to position the second sensor so that the first plane is in the predetermined orientation with respect to the sagittal plane of the user, preferably to the sagittal plane of the user's head, and preferably parallel to the sagittal plane of the user, preferably to the sagittal plane of the user's head. The adjustment and locking elements can be used to immobilise the second sensor with respect to the sagittal plane of the user, preferably to the sagittal plane of the user's head. If the adjustment elements comprise pivoting elements for pivoting the base about a pivoting axis perpendicular to the sagittal plane of the user, preferably to the sagittal plane of the user's head, the adjustment elements can be used to position the second sensor in a position where a plane formed by two axes of the accelerometer of the second sensor is parallel to the frontal plane of the user, preferably to the frontal plane of the user's head. In addition to the sagittal plane of the user, preferably the sagittal plane of the user's head, a second reference plane with respect to the user's nose can therefore be obtained.

In practice and advantageously, when the second holder is positioned on the forehead or nose of the user standing or sitting and looking in a horizontal direction, towards the horizon, the base of the sensor can be pivoted so that the second holder is vertical with respect to the ground.

Thus, the second sensor moves together with the user's nose. This has the advantage that the position of the second sensor represents in real time the orientation of the nose and in particular of the sagittal and frontal planes of the user, preferably the sagittal and frontal planes of the user's head, which define a reference position for the first sensor. It is therefore possible to calculate the relative angles between the first holder and the nose, in particular between the first holder and both the frontal plane and the sagittal plane of the user, preferably between the first holder and both the frontal and sagittal planes of the user's head, to determine even more precisely whether the device is positioned correctly.

“Moves together” means that a movement in space of the user's nose results in a similar movement of the second holder and therefore of the second sensor.

The second holder comprises a fastening device on the user's head and a base for receiving the second orientation sensor, the fastening device comprising at least three bearing zones on the user's face.

For example, the second holder is configured to be in contact with the user's face by means, for example, of two bearing zones on the nose and one bearing zone on the forehead, or two bearing zones on the ears and two bearing zones on the nose. In this way, the second holder is fastened to the user's face in a more stable manner and the positioning of the dispensing device with respect to the nose is monitored more precisely.

The fastening device of the second holder comprises means for centring on the user's nose, for example pads bearing on each side of the nose; and branches configured to bear against the user's ears or an element for pressing against the forehead, for example an elastic headband configured to be pressed around the user's head. The positioning of the second holder is thus particularly intuitive and stable.

When the second holder comprises an elastic headband configured to be pressed around the user's head, the second holder preferably comprises a plate pressed against the forehead and carrying the second sensor. This method of fastening to the forehead is particularly efficient.

The first orientation sensor is configured to measure a data relating to the orientation of the first holder in space along three reference axes, the first holder being configured so that, when it is fastened to the dispensing device, one of the reference axes of the first orientation sensor is in a predetermined orientation with respect to a dispensing axis of the dispensing device, preferably so that one of the reference axes of the first orientation sensor is parallel to, more preferably coincides with, the dispensing axis.

Thus, the orientation given by the first orientation sensor corresponds to the exact orientation of the dispensing axis, which provides a precise information about the orientation of a dispensing head of the dispensing device and simplifies the information processing.

The dispensing head of the dispensing device defines a dispensing axis corresponding to the axis in which the product is delivered. Generally, but not exclusively, the dispensing axis coincides with an axis of the product reservoir, in particular an axis of symmetry of this reservoir.

According to a non-limiting example, the first holder has an axis of symmetry that coincides with the axis of symmetry of the dispensing device when the first holder is fastened to the dispensing device, so that one of the reference axes of the first sensor is in a predetermined position with respect to the dispensing axis of the device, preferably one of the reference axes of the first sensor is parallel to, more preferably coincides with, the dispensing axis.

The first holder comprises activation means configured to be actuated by the fingers of a person and to cooperate with the dispensing device in order to deliver a dose of product, the first orientation sensor being configured to detect the actuation of the activation means in addition to the orientation of the first holder.

Actuation of the activation means causes a movement of at least a part of the dispensing device, for example a first part of a dispensing pump movable with respect to a second part of the dispensing pump. By detecting the actuation of the activation means, it is possible to detect the time when the administration was carried out, therefore the orientation of the device at the time of administration. It is therefore possible to measure the orientation of the dispensing device at the time when the product dispensing was actuated. For example, the first orientation sensor is arranged on the activation means.

As an alternative or in combination, the first orientation sensor comprises a force sensor adapted to detect actuation of the means for activating the dispensing device or handling of the dispensing device. Advantageously, the first holder also comprises a control member, adapted to trigger activation of the control assembly after having detected, by means of the first sensor, the handling of the dispensing device by a user.

According to one embodiment of the invention, the activation means of the first holder are configured to be moved in a direction that coincides with the direction of an axis of the reservoir. For example, the axis of the reservoir is an axis of symmetry of the reservoir, more particularly an axis of the product reservoir passing through the dispensing orifice.

Alternatively, the activation means of the first holder are configured to be moved in a direction that is orthogonal to the direction of an axis of the tank. For example, the axis of the tank is an axis of symmetry of the reservoir, more particularly an axis of the product reservoir passing through the dispensing orifice. Thus, a lateral activation device can be used.

The assembly for controlling the dispensing of a product comprises signalling means, called first signalling means, configured to emit a first signal when the processing system determines that the first holder is positioned in a predetermined orientation with respect to the second holder.

It is therefore possible to provide an information to the user in real time relating to the correct orientation of the dispensing device, so that he can actuate the means for activating the correctly oriented dispensing device and so that a satisfactory quantity of product can be dispensed in the target area. The signal can be a visual, audible or tactile signal or any of their combinations. For example, the signal can be emitted visually on a remote screen.

The assembly for controlling the dispensing of a product comprises signalling means, called second signalling means, configured to emit a second continuous or intermittent signal, for as long as the processing system determines that the first holder is not positioned in a predetermined orientation with respect to the second holder.

The second signalling means then plays a guiding role when positioning the first holder so that product dispensing is only triggered when the device is correctly positioned. The second signalling means can be the same as the first signalling means. For example, an indicator light capable of emitting two different colours depending on the circumstances can be used.

The processing system is configured to determine, when the second holder is fastened to the user's head, according to the data measured by the first and second orientation sensors: a first angle (a) formed between a dispensing axis of the dispensing device and the frontal plane of the user, and/or a second angle (b) formed between a dispensing axis of the dispensing device and the sagittal plane of the user. The predetermined orientation corresponds to a position of the first holder allowing the axis of the dispensing device, called dispensing axis, to form with the frontal plane of the user a first angle (a) of between 25° and 35°, preferably equal to 30°; and with the sagittal plane of the user a second angle (b) of between 5° and 15°, preferably equal to 10°.

The processing system is configured to determine, when the second holder is fastened to the user's head, according to the data measured by the first and second orientation sensors: a first angle (α) formed between a dispensing axis of the dispensing device and the frontal plane of the user's head, and/or a second angle (β) formed between a dispensing axis of the dispensing device and the sagittal plane of the user's head.

Preferably, the predetermined orientation corresponds to a position of the first holder allowing the axis of the dispensing device, called dispensing axis, to form with the frontal plane of the user's head a first angle (α) of between 25° and 35°, preferably equal to 30°; and with the sagittal plane of the user's head a second angle (β) of between 5° and 15°, preferably equal to 10°.

According to the research studies and tests described in the scientific literature, such an inclination in the sagittal plane and the frontal plane of the user, preferably in the sagittal plane and the frontal plane of the user's head, with respect to the user's nose promotes the optimum deposition of product in certain specific areas, in particular the olfactory region in the upper nasal cavity.

Advantageously, the activation means are configured to be locked depending on the information provided relating to the orientation of the first holder with respect to the second holder.

This configuration can be used to lock the activation means when the first holder is not positioned in a predetermined orientation. The advantage of this configuration is to promote optimum administration of the dose of product in the target area. This configuration is even more interesting when the product dispensing device contains a single dose.

Advantageously, the assembly for controlling the dispensing of a product comprises a contact sensor adapted to be arranged on the dispensing head and configured to detect a contact with a predetermined area of the user's face.

It is therefore possible to make an additional check of the optimum positioning of the dispensing device with respect to one of the user's nostrils. In particular, it is possible to check both a contact with a lip of the user and the bottom of the user's nose, the lip possibly being for example the upper lip or the white upper lip, in order to promote satisfactory sagittal positioning. The contact sensor can be a photosensitive sensor or a resistive sensor or a capacitive sensor or an electrical contactor.

The invention also relates to a system for the dispensing of a product comprising a dispensing device, which comprises a product reservoir and a dispensing head, and an assembly for controlling the dispensing of a product into a nasal cavity of a user as described above.

Advantageously, the dispensing device is configured to be fastened to the first holder via fastening means, such as an adhesive system, a clip system, a snap-fit system, shape cooperation between the first holder and the dispensing device such as an insertion fitting.

Advantageously, the system for the dispensing of a product can comprise a disposable part and a reusable part. In this case, the first holder is part of the reusable part and the product reservoir or the dispensing head can be part of the disposable part so that different types of product or dispensing device can be used with the system.

The invention also relates to a first holder for an assembly for controlling the dispensing of a product as described previously.

The invention also relates to a second holder for an assembly for controlling the dispensing of a product as described previously.

The invention also relates to a method for controlling the dispensing of a product into a nasal cavity of a user using an assembly for controlling the dispensing of a product as described previously, comprising the following steps:
step 1: the first holder is fastened to a product dispensing device for nasal pathway,
step 2: the second holder is fastened to forehead or nose of a user, so that a first plane formed by two of the reference axes of the second orientation sensor is located in a predetermined orientation with respect to a sagittal plane of the user, preferably the sagittal plane of the user's head, or with respect to a frontal plane of the user, preferably the frontal plane of the user's head.
step 3: the first orientation sensor and the second orientation sensor measure data, and
step 4: the information processing system determines an information about the orientation of the first holder with respect to the second holder according to the data measured by the first and second orientation sensors.

Advantageously, during step 2 of fastening the second holder to the user's head, the first plane is in a predetermined orientation with respect to the sagittal plane of the user, preferably with respect to the sagittal plane of the user's head, and the angular positioning of the second orientation sensor is adjusted so that a second plane, formed by two of the reference axes of the second orientation sensor and different from the first plane, is located in a predetermined orientation with respect to a frontal plane of the user, preferably with respect to the frontal plane of the user's head.

### BRIEF DESCRIPTION OF THE FIGURES

It will be easier to understand the invention on reading the description below, given solely by way of example and referring to the attached drawings, in which:

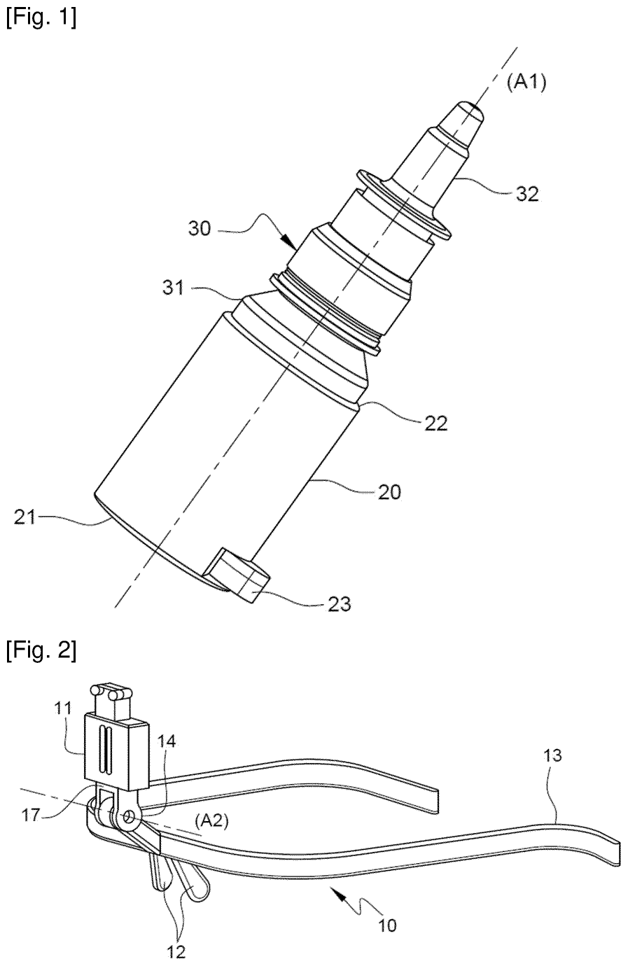

The FIG. 1 is a side view of a product dispensing device for nasal pathway provided with a first holder and a sensor forming part of an assembly for controlling the dispensing of a product into a nasal cavity of a user according to the invention;

The FIG. 2 is a perspective view of a second holder according to a first embodiment, the second holder forming part of an assembly for controlling the dispensing of a product into a nasal cavity of a user;

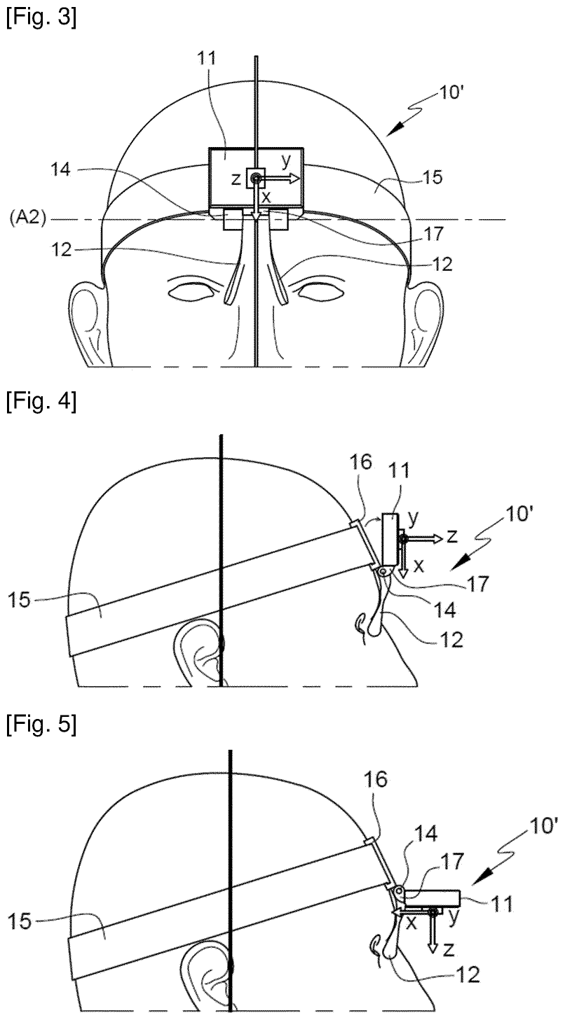

The FIG. 3 is a front view of the second holder according to a second embodiment;

The FIG. 4 is a side view of the second holder of FIG. 3, in a predetermined orientation;

The FIG. 5 is a side view of the second holder of FIG. 3, in another predetermined orientation;

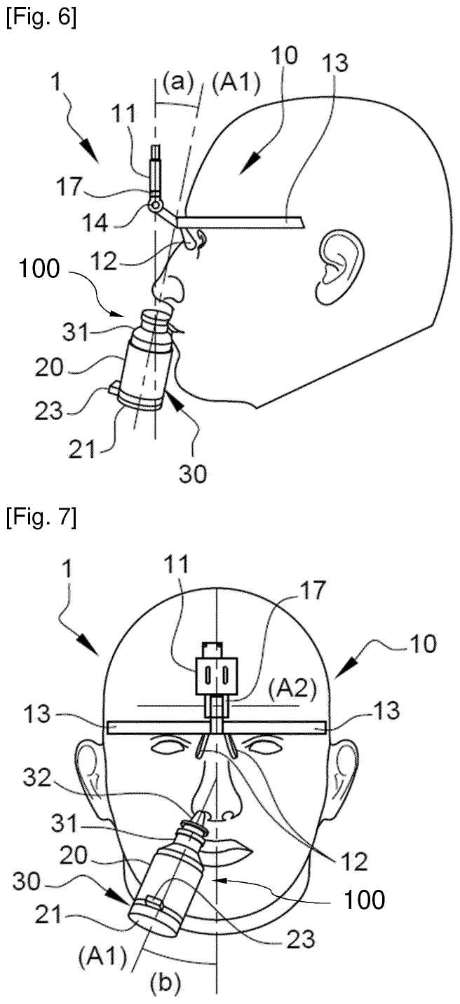

The FIG. 6 is a side view of a system for the dispensing of a product into a nasal cavity of a user comprising the dispensing device provided with the first holder of FIG. 1 and the second holder of FIG. 2;

The FIG. 7 is a front view of the system for the dispensing of a product of FIG. 6;

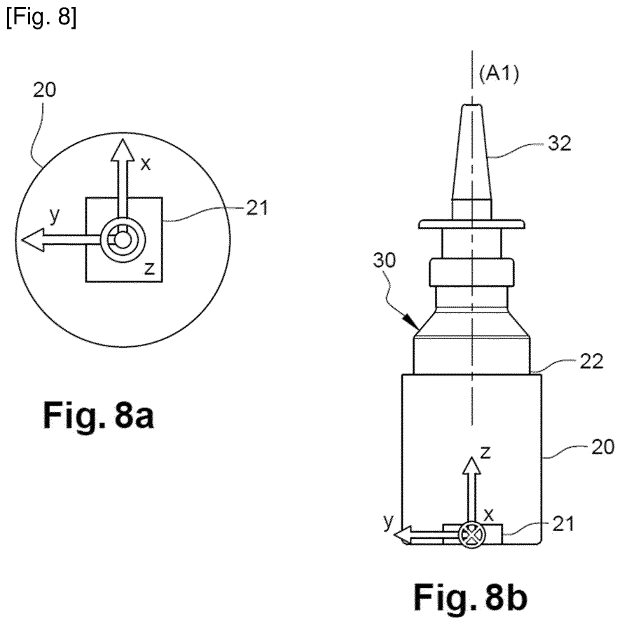

The FIG. 8 is a set of views (FIGS. 8a and 8b) of the first holder of FIG. 1 fastened to the dispensing device.

### DETAILED DESCRIPTION

FIGS. 1 to 2 represent an assembly for controlling the dispensing of a product into a nasal cavity of a user according to a first embodiment. This assembly, also shown on FIGS. 6 and 7, is designated by the general reference 1 and is represented associated with the product dispensing device 30 for nasal pathway on these figures.

More generally, FIGS. 6 and 7 show a system 100 for the dispensing of a product into a nasal cavity of a user comprising the dispensing device 30 and the assembly 1 for controlling the dispensing of a product.

As shown on FIG. 1, the assembly comprises a first holder 20, which carries a first orientation sensor 21. The shape of the first holder 20 is preferably generally cylindrical and hollow, delimiting a housing for receiving the reservoir. In this embodiment, the bottom of the first holder carries the first orientation sensor 21, for example on its outer face. The first holder 20 comprises fastening means 22 for fastening to the dispensing device 30 which are, for example, fastening means of fastening by snap-fastening or by shape cooperation.

The first orientation sensor 21 is configured to measure a data relating to the orientation of the first holder 20 in space along three reference axes.

The first orientation sensor 21 is configured to communicate with an information processing system in order to provide, preferably in real time, the data relating to the orientation of the first holder 20 in space.

The processing system also belongs to the assembly 1.

As shown on FIG. 1, the dispensing device 30 can comprise a cylindrical product reservoir 31 with an axis of symmetry (A1). A dispensing member, such as a pump or a valve, is assembled on the product reservoir 31. The dispensing device 30 further comprises a dispensing head 32 connected to the dispensing member. The dispensing head 32 has a dispensing orifice which is connected to the product reservoir 31 by a channel for the product to be dispensed. In the example shown, the dispensing head 32 comprises a hollow sleeve forming a nasal tip and having an axis of symmetry that coincides with the axis of symmetry (A1) of the product reservoir 31 so that the axis of symmetry (A1) of the product reservoir 31 is also the axis of the dispensing device (A1). In addition, it is advantageous to arrange the dispensing orifice in the axis of the dispensing device (A1).

The first holder 20 is configured so that, when it is fastened to the dispensing device 30, one of the reference axes of the first orientation sensor 21 is in a predetermined orientation with respect to the dispensing axis (A1) of the dispensing device 30, preferably one of the reference axes of the first sensor 21 is parallel to, more preferably coincides with, the dispensing axis (A1).

According to one embodiment of the first holder 20 as shown on FIGS. 8a and 8b, the first orientation sensor 21 is arranged at one of the ends of the first holder 20, for example at the end located opposite the dispensing head 32. The z axis of the first orientation sensor 21 coincides with the dispensing axis of the dispensing device (A1) when the first holder 20 is fastened to the dispensing device 30.

Advantageously, the first holder 20 further comprises activation means 23 configured to be actuated by the fingers of a person and to cooperate with the dispensing device 30 in order to dispense a predetermined dose of product. For example, the activation means 23 are adapted to move the product reservoir 31 with respect to the dispensing head 32 along the dispensing axis (A1) to actuate the dispensing member. As an alternative, the activation means 23 could consist of the bottom of the first holder 20, the outer surface of said bottom forming a bearing surface to move the product reservoir 31 with respect to the dispensing head 32 along the dispensing axis (A1).

Advantageously, the first orientation sensor 21 is adapted to detect the actuation of the activation means 23. Thus, the first orientation sensor 21 can be used to detect the time when the administration was carried out and the orientation of the device at the time of administration. The first orientation sensor 21 is for example adapted to be positioned on the activation means 23 to detect the time when the product was administered.

As an alternative, the first orientation sensor 21 also comprises a force sensor adapted, when the first holder 20 is fastened to the dispensing device 30, to detect actuation of the activation means 23 of the dispensing device 30 and/or the handling of the dispensing device 30. Advantageously, the first holder 20 also comprises a control member, adapted to trigger activation of the assembly for controlling once the first orientation sensor 21 has detected the handling of the dispensing device 30 by a user.

The assembly 1 comprises a second holder 10, 10′, of which a first embodiment is shown on FIG. 2 and on FIGS. 6 and 7. The second holder 10 comprises a fastening device 12, 13 on the user's head, a second orientation sensor 11 and a base 17 for receiving the second sensor 11.

In the embodiment as shown on FIGS. 2, 6 and 7, the fastening device 12, 13 comprises branches 13 configured to bear against the user's ears in order to fasten the reference holder 10 to the user's head. The reference holder 10 further comprises pads 12 intended to bear against each side of the user's nose, in other words on each side of the bone forming the nose, and thus forming centring means 12.

The second orientation sensor 11 is configured to measure a data relating to the orientation of the second holder 10 and in particular of the base 17 in space along three reference axes.

The second orientation sensor 11 is configured to communicate with the processing system in order to provide, preferably in real time, the data relating to the orientation of the second holder 10 in space, and therefore to the orientation of the user's head in space when the second holder 10 and in particular the fastening device 12, 13 is fastened to the user's head and/or face. The first and second orientation sensors 11, 21 used in this invention are Inertial Measurement Units (IMU). Each inertial measurement unit consists of at least one accelerometer and one gyroscope. The accelerometer can be used to measure the linear acceleration of an object along three measurement axes also called reference axes of the sensor and the gyroscope can be used to measure the angular speed along three inclination axes. Regarding the accelerometer, the three axes form an orthonormal reference frame as well as three orthogonal planes. Generally, the inclination axes of the gyroscope coincide with the measurement axes of the accelerometer in an inertial measurement unit.

In this invention, so that the sensors can provide precise information, the axes or the orthogonal planes must be adjusted with respect to a reference frame. Thus, adjustment elements 14 for adjusting the position of the base 17 and therefore the second orientation sensor 11 with respect to the fastening device 12, 13 are provided on the second holder 10. The adjustment elements 14 comprise pivoting elements for pivoting the second orientation sensor 11 about a pivoting axis (A2) perpendicular to the sagittal plane of the user, preferably to the sagittal plane of the user's head, as shown on FIG. 3. The adjustment elements 14 can be associated with locking elements for locking the position of the base 17 so that the base 17 and the second orientation sensor 11 can be immobilised in a predetermined position. Adjustment of the base 17 will be described below.

FIGS. 3 to 5 show a second holder 10′ according to a second embodiment, in which the fastening device 12, 15 comprises an elastic headband 15 forming a pressing element 15 designed to be pressed around the user's head. In this embodiment, the elastic headband 15 further comprises pads 12 similar to those of the first embodiment and a plate 16 configured to bear against the user's forehead. As in the first embodiment, the second holder 10′ also comprises adjustment 14 and locking elements used to adjust the angular position of the second orientation sensor 11 about the pivoting axis (A2).

According to one embodiment, the fastening device 12, 13, 15 comprises at least three separate bearing zones on the user's face in order to position the second orientation sensor 11 and in particular its reference axes in a predetermined position with respect to the sagittal and/or frontal planes of the user, preferably with respect to the sagittal and/or frontal planes of the user's head.

As shown on FIGS. 3 and 4, when the second holder 10′ is fastened to the user's head, the second orientation sensor 11 is located in a predetermined orientation with respect to the sagittal plane and frontal plane of the user, preferably with respect to the sagittal plane and frontal plane of the user's head: a first plane formed by two of the reference axes of the second orientation sensor 11 is located, when the second holder 10′ is fastened to the user's head, in a predetermined orientation with respect to the sagittal plane of the user, preferably with respect to the sagittal plane of the user's head, and a second plane formed by two of the reference axes of the second orientation sensor 11 and different from the first plane, is located, when the second holder 10′ is fastened to the user's head, in a predetermined orientation with respect to the frontal plane of the user, preferably with respect to the frontal plane of the user's head.

Advantageously, the first plane extends parallel to the sagittal plane of the user, preferably to the sagittal plane of the user's head. More precisely, in the example shown, the three axes of the accelerometer of the second orientation sensor 11 respectively coincide with the three axes of the gyroscope of the same sensor. In this case, the three axes are called the x, y and z axes but the orientation of these axes is arbitrary and can be changed. The pressing of the headband 15 on the head or the branches 13 bearing against the user's ears, as well as the positioning of the centring means 12 on the nose ensure that the plane (x, z) formed by the x and z axes is substantially parallel to or coincides with the sagittal plane of the user, preferably with the sagittal plane of the user's head, when the second holder 10, 10′ is fastened to the user's head. The plane formed by the x and z axes is thus substantially aligned with the axis of the nose. After positioning the second holder 10, 10′ on the user's head and before using the system 100 for the dispensing of a product for the first time, the angular positioning of the second orientation sensor 11 with respect to the frontal plane of the user, preferably with respect to the frontal plane of the user's head, must be adjusted. To do this and as shown on FIG. 4, the user looks in front far ahead, towards the horizon, in a substantially horizontal plane. The adjustment elements 14 are then used to pivot the second orientation sensor 11 until the z axis is substantially perpendicular to the frontal plane of the user, preferably to the frontal plane of the user's head. Visually, it is possible to assess the parallelism between the frontal plane of the user, preferably the frontal plane of the user's head, and a plane formed by the two other x and y axes, or the verticality of one of the faces of the second orientation sensor 11 when it has the shape of a rectangular parallelepiped. This position of the second orientation sensor 11 then corresponds to a reference, or predetermined, position where the x axis of the second orientation sensor 11 is used as reference axis so that the processing system can calculate a sagittal angle (a) between the dispensing axis (A1) and the frontal plane of the user, preferably the frontal plane of the user's head, and a frontal angle (b) between the dispensing axis (A1) and the sagittal plane of the user, preferably the sagittal plane of the user's head. The second orientation sensor 11 is held in this position by the adjustment elements 14 and the locking elements to keep the adjustment during all subsequent uses of the system for the dispensing of a product. In this position the second plane (formed by the x and y axes) is in a predetermined orientation with respect to the frontal plane of the user, preferably with respect to the frontal plane of the user's head, and preferably extends parallel to the frontal plane of the user, preferably to the frontal plane of the user's head.

The designation of the three x, y and z axes does not form a limitation regarding the orientation of the sensors and is arbitrary. According to an adjustment variant as shown on FIG. 5, the second orientation sensor 11 can be positioned in another reference or predetermined position in which the second orientation sensor 11 has pivoted 90° about the pivoting axis (A2) with respect to the position shown on FIG. 4. In this position, the x axis is perpendicular to the frontal plane of the user, preferably to the frontal plane of the user's head, and the z axis is used to calculate the sagittal angle (a) and the frontal angle (b).

FIGS. 6 and 7 show the assembly 1 for controlling the dispensing of a product comprising the first holder 20 and the second holder 10 according to the first embodiment shown on FIG. 2, the first holder 20 being positioned according to a predetermined orientation with respect to the second holder 10. As shown on FIGS. 6 and 7, the predetermined orientation corresponds to an orientation of the first holder 20 where the axis of the dispensing device (A1) forms with the frontal plane of the user a first angle (a) which is about 30°, preferably with the frontal plane of the user's head a first angle (a) which is about 30°. It is therefore a predetermined sagittal angle. With this same orientation, the axis of the dispensing device (A1) forms with the sagittal plane of the user a second angle (b) which is about 10°, preferably with the sagittal plane of the user's head a second angle (β) which is about 10°. It is a predetermined frontal angle.

The first angle (a) is called a sagittal angle since it is measured in the sagittal plane of the user, whereas the second angle (b) is called a frontal angle since it is measured in the frontal plane.

The first angle (α) is called a sagittal angle since it is measured in the sagittal plane of the user's head, whereas the second angle (β) is called a frontal angle since it is measured in the frontal plane of the user's head.

According to a variant of the invention, the predetermined orientation required for the first holder 20 with respect to the second holder 10, 10′ and in particular to the base 17 is such that the sagittal angle is between 25° and 35°, preferably equal to 30°; and that the frontal angle is between 5° and 15°, preferably equal to 10°.

The first and second orientation sensors 11, 21 communicate with the processing system which can comprise an electronic module such as a printed circuit board (PCB). The electronic module comprises for example a microprocessor containing software for processing the data provided by the first orientation sensor 21 and the second orientation sensor 11 to determine an information about the orientation of the first orientation sensor 21, and therefore of the first holder 20 with respect to the second orientation sensor 11 and therefore to the second holder 10, 10′ and in particular to the base 17. Each first and second orientation sensor 21, 11 is adapted to provide data relating to its orientation with respect to the terrestrial reference frame and the processing system is adapted to calculate, according to said data, the orientation of the first orientation sensor 21 in the reference frame of the second orientation sensor 11, in other words with respect to the second orientation sensor 11. By comparing the relative angles between the first orientation sensor 21 and the second orientation sensor 11, the processing system calculates angles between the first orientation sensor 21 and the second orientation sensor 11 and in particular the sagittal angle (a) and the frontal angle (b) between the product dispensing axis (A1) and a reference axis of the second orientation sensor 11 whose position is defined with respect to the sagittal and frontal planes of the user, preferably with respect to the sagittal and frontal planes of the user's head, as shown on FIGS. 6 and 7. Thus, since the orientation of the first holder 20 and therefore of the first orientation sensor 21 with respect to the dispensing device 30 and in particular to the dispensing axis (A1) is known, as well as the orientation of the second holder 10, 10′ and therefore of the second orientation sensor 11 with respect to the sagittal and frontal planes of the user, preferably with respect to the sagittal and frontal planes of the user's head, the sagittal and frontal angles can be determined. Thanks to the second holder 10, 10′, the orientation of the reference axis of the second orientation sensor 11 with respect to the user's nose is repeatable, regardless of the user's position.

Advantageously, the processing system is embedded on the second holder 10, 10′ or on the first holder 20. According to a variant of the invention, the processing system is separate from the first holder 20 and the second holder 10, 10′. It can, for example, be integrated into a multifunction mobile phone, also called a smartphone, or any other suitable electronic device.

Advantageously, the processing system is adapted to lock the activation means 23 according to the information provided relating to the orientation of the first holder 20 with respect to the second holder 10, 10′. This configuration can be used to lock the activation means 23 when the first holder 20 is not positioned in the predetermined orientation.

In the embodiments described, when the second holder 10, 10′ is fastened to the user's head, the second orientation sensor 11 is located in a predetermined orientation with respect to the sagittal plane and the frontal plane of the user, preferably with respect to the sagittal plane and the frontal plane of the user's head. However, in other embodiments of the invention, the second holder is configured to be fastened to the user's forehead or nose, so that the second orientation sensor 11 is located in a predetermined orientation with respect to at least one of the sagittal and frontal planes of the user, preferably with respect to at least one of the sagittal and frontal planes of the user's head. These embodiments can be used to determine at least one of the sagittal and frontal angles and therefore to obtain a reliable and efficient control assembly for detecting the orientation of the dispensing device with respect to one of the user's nostrils.

A method for controlling the dispensing of a product into a nasal cavity of a user using the assembly 1 for controlling the dispensing of a product as described previously and comprising, for example, a second holder 10 according to the first embodiment of the invention will be described below. The method comprises the following steps:
step 1: the first holder 20 is fastened to a product dispensing device 30 for nasal pathway.
step 2: the second holder 10, 10′ is fastened to the forehead or nose of a user, so that a first plane formed by two of the reference axes of the second orientation sensor 11 is located in a predetermined orientation with respect to a sagittal plane of the user, preferably with respect to the sagittal plane of the user's head, or with respect to a frontal plane of the user, preferably with respect to the frontal plane of the user's head. Advantageously, during step 2, the second holder 10, 10′ is fastened to the user's head so that the first plane is parallel to the sagittal plane of the user, preferably to the sagittal plane of the user's head. For example, the branches 13 of the fastening device 12, 13 bear against the user's ears and the pads 12 bear against each side of the user's nose. Advantageously, during step 2, the position of the base 17 is adjusted using the adjustment elements 14 to pivot the base 17 and therefore the second sensor 11 about the pivoting axis (A2) so that the second plane is parallel to the frontal plane of the user, preferably to the frontal plane of the user's head. To do this, the user looks towards the horizon in a substantially horizontal plane of vision and the user or a practitioner pivots the base 17 to position the base 17 vertically with respect to the ground, then locks the position of the base 17 using the locking elements.
step 3: the first orientation sensor 21 and the second orientation sensor 11 measure data. Advantageously, the measurement is triggered only when the handling of the dispensing device 30 by a user is detected.
step 4: the data processing system determines an information about the orientation of the first holder 20 with respect to the second holder 10, 10′ according to the data measured by the first orientation sensor 21 and the second orientation sensor 11.

When it is for example implemented for each dispensing device of a set of dispensing devices to be tested during “user studies”, such a method helps to identify which dispensing device obtains the positioning required to achieve efficient product dispensing in the target area.

Advantageously, prior to step 1, the method comprises a step of initialising the first and second orientation sensors 21, 11 during which the first and second orientation sensors 21, 11 and in particular the first and second holders 20, 10, 10′ are positioned parallel to each other so that the respective reference axes of the sensors are parallel to define a common reference for the subsequent measurement of the orientation of the sensors. The operation of the first and second orientation sensors 21, 11 is then activated or the sensors are reinitialised to store this common reference.

The invention is not limited to the embodiments described and other embodiments will be clearly apparent to those skilled in the art. In particular, a first signalling means can be used to emit a first signal when the first holder 20 is positioned in the required predetermined orientation with respect to the second holder 10, 10′. A second signalling means can also be used to emit a second continuous or intermittent signal, for as long as the first holder 20 is not positioned in the predetermined orientation. Thus, after step 4, the processing system is for example adapted to transmit, according to the orientation of the first holder 20 with respect to the second holder 10, 10′, information to help the user position the dispensing device 30 correctly, for example when used during a clinical phase, or at home.

### List of References

1: assembly
10, 10′: second holder
11: second orientation sensor
12: centring means, pads
13: branches
14: adjustment elements
15: pressing element, headband
16: plate
17: base
20: first holder
21: first orientation sensor
22: fastening means
23: activation means
30: dispensing device
31: product reservoir
32: dispensing head
100: system for the dispensing of a product
A1: device axis, dispensing axis
A2: pivoting axis
a: first angle, sagittal angle
b: second angle, frontal angle

1. An assembly for controlling the dispensing of a product into a nasal cavity of a user, comprising:
a first holder configured to be fastened to a product dispensing device for a nasal pathway, and comprising a first orientation sensor configured to measure first data relating to an orientation of the first holder in space,
a second holder configured to be fastened to the head of a user, and comprising a second orientation sensor configured to measure second data relating to an orientation of the second holder in space along three reference axes, and
an information processing system configured to receive the first data measured by the first orientation sensor and the second data measured by the second orientation sensor and to determine information about an orientation of the first holder with respect to the second holder according to said first and second data,
the second holder being configured to be fastened to the user's forehead or nose, so that a first plane formed by two of the three reference axes of the second orientation sensor is located, when the second holder is fastened to the user's head, in a predetermined orientation with respect to a sagittal plane of the user or with respect to a frontal plane of the user.

2. The assembly according to claim 1, wherein the predetermined orientation of the first plane corresponds to an orientation parallel to the sagittal plane of the user or to the frontal plane of the user.

3. The assembly according to claim 1, wherein the second holder is configured to be fastened to the user's forehead or nose, so that:
the first plane is located in a predetermined orientation with respect to the sagittal plane of the user, and
a second plane, formed by two of the reference axes of the second orientation sensor and different from the first plane, is located in a predetermined orientation with respect to a frontal plane of the user.

4. The assembly according to claim 1, wherein the second orientation sensor comprises an accelerometer configured to measure the linear acceleration along three measurement axes, the measurement axes corresponding to the reference axes of the second orientation sensor.

5. The assembly according to claim 1, wherein the second holder comprises:
a fastening device on the user's head,
a base for receiving the second orientation sensor,
adjustment elements for adjusting the position of the base, with respect to the fastening device, and locking elements for locking the position of the base so that the position of the second orientation sensor with respect to the sagittal plane can be adjusted and locked once the fastening device is fastened to the head of a user,
the adjustment elements preferably comprising pivoting elements for pivoting the base about a pivoting axis perpendicular to the sagittal plane of the user.

6. The assembly according to claim 1, wherein the second holder comprises a fastening device on the user's head and a base for receiving the second orientation sensor, the fastening device comprising at least three bearing zones on the user's face.

7. The assembly according to claim 6, wherein the fastening device comprises:
a centring element for centring on the user's nose, for example pads (12) bearing on each side of the nose, and
branches configured to bear against the user's ears or an pressing element for pressing against the forehead, for example an elastic headband configured to be pressed around the user's head.

8. The assembly according to claim 1, wherein the first orientation sensor is configured to measure a data relating to the orientation of the first holder in space along three reference axes, the first holder being configured so that, when the first holder is fastened to the dispensing device, one of the reference axes of the first orientation sensor is in a predetermined orientation with respect to a dispensing axis of the dispensing device, preferably so that one of the reference axes of the first orientation sensor is parallel to, more preferably coincides with, the dispensing axis.

9. The assembly according to claim 1, wherein the first holder comprises activation means configured to be actuated by the fingers of a person and to cooperate with the dispensing device in order to dispense a dose of product and wherein the first orientation sensor is configured to detect the actuation of the activation means in addition to the orientation of the first holder.

10. The assembly according to claim 1, comprising a first signalling device configured to emit a first signal when the processing system determines that the first holder is positioned in a predetermined orientation with respect to the second holder.

11. The assembly according to claim 1 comprising a second signalling device configured to emit a second continuous or intermittent signal, for as long as the processing system determines that the first holder is not positioned in a predetermined orientation with respect to the second holder.

12. The assembly according to claim 1, wherein the processing system is configured to determine, when the second holder is fastened to the user's head, according to the data measured by the first orientation sensor and the second orientation sensor:
a first angle (a) formed between a dispensing axis of the dispensing device and the frontal plane, and/or
a second angle (b) formed between a dispensing axis of the dispensing device and the sagittal plane.

13. A system for the dispensing of a product into a nasal cavity of a user comprising a product dispensing device for nasal pathway and an assembly according to claim 1.

14. A method for controlling the dispensing of a product into a nasal cavity of a user using an assembly according to claims 1 comprising the following steps:
step 1: the first holder is fastened to a product dispensing device for nasal pathway,
step 2: the second holder is fastened to the forehead or nose of a user, so that a first plane formed by two of the reference axes of the second orientation sensor is located in a predetermined orientation with respect to a sagittal plane of the user, or with respect to a frontal plane of the user,
step 3: the first orientation sensor and the second orientation sensor measure the first and second data, and
step 4: the information processing system determines an information about the orientation of the first holder with respect to the second holder according to the first and second data measured by the first orientation sensor and the second orientation sensor.

15. A method according to claim 14, wherein during step 2 of fastening the second holder to the user's head, the first plane is in a predetermined orientation with respect to the sagittal plane of the user, and the angular positioning of the second orientation sensor is adjusted so that a second plane, formed by two of the reference axes of the second orientation sensor and different from the first plane, is located in a predetermined orientation with respect to a frontal plane of the user.
